(19) United States
(12) Patent Application Publication
(10) Pub. No.: US 2006/0084656 A1
(43) Pub. Date: Apr. 20, 2006

(54) Compositions and methods for intranasal, buccal, sublingual and pulmonary delivery of varenicline

(75) Inventors: Carl B. Ziegler, East Lyme, CT (US); Barbara A. Johnson, Niantic, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 11/251,171

(22) Filed: Oct. 14, 2005

Related U.S. Application Data
(60) Provisional application No. 60/619,094, filed on Oct. 15, 2004.

(51) Int. Cl. A61K 31/498

(52) U.S. Cl. 514/249

(57) ABSTRACT

A composition for nasal administration comprising varenicline or its pharmaceutically acceptable salt and at least one excipient. The invention also provides a composition for buccal administration comprising varenicline or its pharmaceutically acceptable salt and at least one excipient to form a solid dosage form, wherein the solid dosage form disintegrates in an oral cavity at body temperature and may adhere to body tissue of the oral cavity; a composition for pulmonary administration comprising varenicline or its pharmaceutically acceptable salt and at least one excipient; and, a method for reducing nicotine addiction, aiding in the cessation of, or lessening of tobacco use in a subject.

### CROSS REFERENCE TO RELATED APPLICATIONS

1. Field of the Invention

The present invention relates to pharmaceutical compositions for medicinal uses thereof.

2. Background Art

Varenicline has the structure:

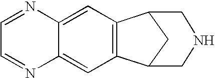

Varenicline is also known as 5,8,14-triazatetracyclo[10.3.1.0^{2,11}.0^{4,9}-hexadeca-2(11),3,5,7,9-pentaene or 7,8,9,10-tetrahydro-6,10-methano-6H-pyrazino[2,3-h][3]-benzazepine. Varenicline and pharmaceutically acceptable acid addition salts thereof are referred to in International Patent Publication WO 99/35131, published Jul. 15, 1999, the contents of which are incorporated herein by reference.

Varenicline binds to neuronal nicotinic acetylcholine specific receptor sites and is useful in modulating cholinergic function. Accordingly, this compound is useful in the treatment of various conditions or diseases including, but not limited to, inflammatory bowel disease (including, but not limited to, ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome.

Varenicline is a highly potent compound such that dosage forms are necessarily highly diluted with excipients. The excipients provide dosage forms with adequate stability, while also providing for such desirable features as controlling the drug dissolution (e.g., either fast dissolving or slow dissolving in a controlled-release system as described in co-pending applications U.S. Patent Publication No. 2003-0180360 A1, published Sept. 25, 2003, and Ser. No. 10/848,464, filed May 18, 2004, the contents of which are hereby incorporated by reference in their entirety), masking bad taste, and providing appropriate properties for preparation of the dosage form (i.e., compression properties for tablets). Finally, because of the high dilution with excipients, reactivity of varenicline with the excipients themselves or with trace impurities (i.e., degradants) of the excipients can be especially problematic.

There are advantages of delivering varenicline via intranasal, buccal, or pulmonary routes. For example, relative to an oral dosage form such as a tablet or capsule, intranasal, buccal, sublingual or pulmonary delivery of varenicline provides for rapid absorption, faster onset of therapeutic action and avoidance of liver first pass metabolism. For patients who have difficulty in swallowing tablets, capsules or other solids, or those who have intestinal failure, the nasal, buccal, sublingual and pulmonary delivery route options of varenicline are preferred. Delivery of varenicline or its suitable pharmaceutical salts, herein referred to as the active ingredient, can be achieved in an intranasal, buccal, sublingual and pulmonary composition.

Accordingly, there is a need for providing dosage forms of varenicline having immediate release formulations, which can be administered via intranasal, buccal, and pulmonary routes.

### SUMMARY OF THE INVENTION

The present invention provides a composition for nasal administration comprising varenicline or its pharmaceutically acceptable salt and an effective amount of an absorption-promoting agent to promote nasal absorption of the varenicline or its pharmaceutically acceptable salt after nasal administration of the composition thereof. Additionally, the present invention provides a composition for buccal administration comprising varenicline or its pharmaceutically acceptable salt and at least one excipient to form a solid dosage form with the varenicline or its pharmaceutically acceptable salt, wherein the solid dosage form disintegrates in an oral cavity at body temperature and adheres to body tissue of the oral cavity via direct adhesion to tissue or entrapment of the dosage form as in between the gum and inner cheek. The present invention also provides a composition for sublingual administration comprising varenicline or its pharmaceutically acceptable salt and at least one excipient to form a solid dosage form with the varenicline or its pharmaceutically acceptable salt, wherein the solid dosage form disintegrates in an oral cavity at body temperature under the tongue. Further, the present invention provides a composition for pulmonary administration comprising varenicline or its pharmaceutically acceptable salt and at least one excipient combined with the varenicline or its pharmaceutically acceptable salt. Finally, the present invention provides a method for reducing nicotine addiction, aiding in the cessation of, or lessening of, tobacco use in a subject.

### DETAILED DESCRIPTION OF THE INVENTION

Generally, the present invention provides various compositions and related methods for intranasal, buccal, and pulmonary delivery of varenicline or its pharmaceutically acceptable salts.

The present invention utilizes varenicline or its pharmaceutically acceptable salt as the active ingredient. Varenicline can be used per se or in the form of its pharmaceutically acceptable salt, solvate and/or hydrate. Although any pharmaceutically acceptable form of varenicline can be used in connection with the present invention, it is preferable to use a salt form of the drug. A particularly preferred salt form of the drug is the L-tartrate salt.

In particular, the present invention provides a method for reducing nicotine addiction or aiding in the cessation or lessening of tobacco use in a subject. The method includes steps of administering to a subject an amount of the immediate-release varenicline that is effective in reducing nicotine addiction or aiding in the cessation or lessening of tobacco use via administration via the intranasal, buccal, sublingual or pulmonary routes.

The present invention can be used to treat disorders or conditions including, but not limited to, inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions; dependencies on, or addictions to, nicotine, tobacco products, alcohol, benzodiazepines, barbiturates, opioids or cocaine; headache, stroke, traumatic brain injury (TBI), obsessive-compulsive disorder (OCD), psychosis, Huntington's Chorea, tardive dyskinesia, hyperkinesia, dyslexia, schizophrenia, multi-infarct dementia, age related cognitive decline, epilepsy, petit mal absence epilepsy, senile dementia of the Alzheimer's type (AD), Parkinson's disease (PD), attention deficit hyperactivity disorder (ADHD), Tourette's Syndrome, and any other similar disorder or condition known to those of skill in the art.

The term “immediate-release form,” “IR form,” or “immediate-release (IR) FDDF” as used herein means a dosage form which when taken orally substantially provides the drug in a form available to be absorbed into the systemic circulation within about one hour or less. The immediate release form of the present invention can further include a glidant, disintegrant, and/or a lubricant.

The term “intranasal delivery” or “nasal delivery” as used herein means a method for drug absorption through and within the nose.

The term “pulmonary delivery” as used herein means a method for drug absorption through the lungs of an individual.

The term “buccal delivery” as used herein means a method for drug absorption through the buccal (i.e., inner cheek) tissue.

The term “sublingual delivery” as used herein means delivery of the active compound of the present invention across any tissue under the tongue.

The term “transmucosal delivery” as used herein means delivery of the active compound of the present invention across any mucosal membrane.

The term “varenicline,” as used herein means the drug that binds to neuronal nicotinic acetylcholine specific receptor sites, and is useful in modulating cholinergic function. Varenicline has the general formula of:

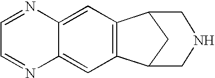

Varenicline includes the parent drug and all pharmaceutically acceptable salts and prodrugs thereof. The parent drug of varenicline is described in International Patent Publication WO 99/35131, published Jul. 15, 1999, the contents of which are incorporated herein by reference in their entirety. In any of the embodiments, varenicline or any of its pharmaceutically acceptable salts, solvates and/or hydrates can be used. Procedures for making varenicline are described in U.S. Pat. No. 6,410,550, the contents of which are incorporated herein by reference in their entirety. The resolution of racemic mixtures of varenicline is described in WO01/62736, which is also incorporated herein by reference in its entirety.

The term “mgA” refers to the number of milligrams of active drug based on the free base form of the drug.

The term “pharmaceutically acceptable” means the substance or composition must be compatible chemically, physically, and/or toxicologically, with the other ingredients comprising a formulation, and/or the mammal being treated therewith.

The term “pharmaceutically acceptable salt” means non-toxic acid addition salts derived from inorganic and organic acids. Suitable salt derivatives include, but are not limited to, halides, thiocyanates, sulfates, bisulfates, sulfites, bisulfites, arylsulfonates, alkylsulfates, phosphonates, monohydrogen-phosphates, dihydrogenphosphates, metaphosphates, pyrophosphonates, alkanoates, cycloalkylalkanoates, arylalkonates, adipates, alginates, aspartates, benzoates, fumarates, glucoheptanoates, glycerophosphates, lactates, maleates, nicotinates, oxalates, palmitates, pectinates, picrates, pivalates, succinates, tartarates, citrates, camphorates, camphorsulfonates, digluconates, trifluoroacetates, and the like.

The term “active ingredient” means a therapeutically active compound (i.e., varenicline) as well as any prodrugs thereof and pharmaceutically acceptable salts, hydrates, and solvates of the compound and the prodrugs. The term “other ingredients” means any excipients, diluents, binders, lubricants, glidants, disintegrants, carriers, surfactants, flavors and mixtures thereof that are formulated with varenicline or any prodrugs thereof and pharmaceutically acceptable salts, hydrates, and solvates of this drug

The term “appropriate period of time” or “suitable period of time” means the period of time necessary to achieve a desired effect or result. For example, a mixture can be blended until a potency distribution is reached that is within an acceptable range for a given application or use of the blended mixture.

The term “unit dose,” “unit dosage,” or “unit dosage form” means a physically discrete unit that contains a predetermined quantity of active ingredient calculated to produce a desired therapeutic effect. The dosage form can be in the form including, but not limited to, tablets, a bioadhesive patch or film, lozenges, hard candies, wafers, lollipops, sprays, gums, pills, pellets, spheres, and other forms known to those of skill in the art.

The term “effective amount,” as used herein means the amount determined by such considerations as are known in the art of reducing nicotine addiction or aiding in the cessation or lessening of tobacco use in an individual, wherein it must be effective to provide measurable relief in treated individuals such as exhibiting improvements including, but not limited to, more rapid recovery, improvement or elimination of symptoms or reduction of complications, lack of dependency upon nicotine-containing compounds, lack of desire towards nicotine-containing compounds, or other measurements as appropriate and known to those skilled in the medical arts.

The present invention has numerous embodiments. In any of the embodiments, pharmaceutical compositions of varenicline can be desirably administered in doses ranging from about 0.1 mgA up to about 6 mgA per day (where mgA refers to mg of active drug based on the free base form of the drug), more preferably from about 0.5 to 4 mgA/day, and most preferably from about 1 to 4 mgA per day in single or divided doses. Variations in such dosages, however, necessarily occur depending upon the weight and condition of the subject being treated. Depending on individual responses, dosage levels below the lower limit of the aforesaid range can be more than adequate, while in other cases still larger doses can be employed without causing any harmful side effects. The final pharmaceutical composition is processed into a unit dosage form and then packaged for distribution. The processing step varies depending upon the particular unit dosage form. For example, a tablet is generally compressed under pressure into a desired shape. Those of skill in the art are well aware of the procedures used for manufacturing the various unit dosage forms.

In any embodiments of the present invention, the active blend of a dosage form generally includes one or more pharmaceutically acceptable excipients, carriers, diluents, binders, lubricants, glidants, disintegrants or flavors used and depends upon the purpose for which the active ingredient is being applied. In general, intranasal, buccal, sublingual or pulmonary formulations are made of other ingredients including, but not limited to, excipients, diluents, binders, lubricants, glidants, disintegrants, carriers, flavors and mixtures thereof.

Buccal and Sublingual Dosage Forms

For buccal and sublingual dosage forms acceptable other ingredients include but are not limited to starch, mannitol, kaolin, calcium sulfate, inorganic salts (e.g., sodium chloride), powdered cellulose derivatives, tribasic calcium phosphate, calcium sulfate, magnesium carbonate, magnesium oxide, poloxamers such as polyethylene oxide, hydroxypropyl methylcellulose, anionic excipients, cationic excipients, zwitterionic excipients (See, U.S. Pat. No. 6,436,950, which is incorporated herein by reference in its entirety), polymeric hydrogel, powder microsphere mucoadhesive compositions, thiolated polymeric excipients, polycationic material, chitosan, cross-linked starches, fats, carbohydrates, polyols, buffers, phosphate buffers, acetate buffers, methocel, sodium chloride, water, lactic acid, benzalkonium chloride, demineralized water, cellulose, microcrystalline cellulose, hydroxypropyl cellulose, hydrogenated vegetable oil, flavoring agents, phospholipids, xylitol, cacao, combinations thereof, and other similar excipients known to those of skill in the art.

Preferred other ingredients that are diluents such as microcrystalline cellulose (e.g., Avicel® PH200, PH102 or PH101 available from FMC Pharmaceutical, Philadelphia, Pa.) and calcium phosphate dibasic, or dicalcium phosphate, (e.g. A-Tab® available from Rhodia, Chicago Heights, Ill.). The mean particle size for the microcrystalline cellulose generally ranges from about 90 μm to about 200 μm. Suitable grades of dicalcium phosphate include anhydrous (about 135 to 180 μm mean, available from PenWest Pharmaceuticals Co., Patterson, N.Y. or Rhodia, Cranbury, N.J.), and dihydrate (about 180 μm, available from PenWest Pharmaceuticals Co., Patterson, N.Y. or Rhodia, Cranbury, N.J.). Generally, the microcrystalline cellulose is present in an amount from about 10 wt % to about 70 wt % and the dicalcium phosphate is present in an amount from about 10 wt % to about 50 wt %. More preferably, microcrystalline cellulose is present in an amount of about 30-70 wt % and the dicalcium phosphate is present in an amount of about 20-40 wt %. Finally, examples of glidants include, but are not limited to, silicon dioxide, talc, cornstarch, combinations thereof, and any other similar glidants known to those of skill in the art.

If desired, a binder may be added. Suitable binders include substances such as celluloses (e.g., cellulose, methylcellulose, ethylcellulose, hydroxypropyl cellulose and hydroxymethylcellulose), polypropylpyrrolidone, polyvinylprrolidone, gelatin, gum arabic, polyethylene glycol, starch, natural and synthetic gums (e.g., acacia, alginates, and gum arabic) and waxes.

A lubricant is typically used in a tablet formulation to prevent the tablet and punches from sticking in the die. Suitable lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc and zinc stearate. A preferred lubricant is magnesium stearate. The magnesium stearate is generally present in an amount from about 0.25 wt % to about 4.0% wt %.

Other Ingredients such as disintegrants may also be added to the composition to break up the dosage form and release the compound. Suitable disintegrants include sodium starch glycolate, sodium carboxymethyl cellulose, calcium carboxymethyl cellulose, croscarmellose sodium, polyvinylpyrrolidone, methyl cellulose, microcrystalline cellulose, powdered cellulose, lower alkyl-substituted hydroxypropyl cellulose, polacrilin potassium, starch, pregelatinized starch and sodium alginate. Of these, croscarmellose sodium and sodium starch glycolate are preferred, with croscarmellose sodium being most preferred. The croscarmellose sodium is generally present in an amount from about 0.5 wt % to about 6.0 wt %. The amount of disintegrant included in the dosage form will depend on several factors, including the properties of the dispersion, the properties of the porosigen (discussed below), and the properties of the disintegrant selected. Generally, the disintegrant will comprise from 1 wt % to 15 wt %, preferably from 1 wt % to 10 wt % of the dosage form.

Other ingredients such as glidants include silicon dioxide, talc and cornstarch.

Intranasal Dosage Forms

For intranasal dosage forms acceptable other ingredients include, but are not limited to carriers and excipients such as but not limited to ion-exchange microspheres which carry suitable anionic groups such as carboxylic acid residues, carboxymethyl groups, sulphopropyl groups and methylsulphonate groups. Ion-exchange resins (cation exchangers) can also be used. Chitosan, which is partially deacetylated chitin, or poly-N-acetyl-D-glucosamine, or a pharmaceutically acceptable salt thereof such as hydrochloride, lactate, glutamate, maleate, acetate, formate, propionate, malate, malonate, adipate, succinate.

Suitable other ingredients for use as non-ion-exchange microspheres include starch, gelatin, collagen and albumin.

The composition may further comprise an appropriate acid selected from the group consisting of hydrochloric acid, lactic acid, glutamic acid, maleic acid, acetic acid, formic acid, propionic acid, malic acid, malonic acid, adipic acid, succinic acid

Excipients to enhance absorption may also be used and further include phospholipids, e.g. phosphatidylglycerol or phosphatidylcholines; or lysophosphatidyl derivatives, e.g. lysolecithin, lysophosphatidyl-ethanolamine, lysophosphatidylcholine, lysophosphatidylglycerol, lysophosphatidylserine, lysophosphatidic acid, cyclodextrins. Gelling excipients or viscosity-increasing excipients may be added.

Other ingredients such as diluents are cellulose, microcrystalline cellulose, hydroxypropyl cellulose, starch, hydroxypropylmethyl cellulose and the like

The intranasal compositions may also contain one or more preservatives selected from quaternary ammonium salts such as lauralkonium chloride, benzalkonium chloride, benzododecinium chloride, cetyl pyridium chloride, cetrimide, domiphen bromide; alcohols such as benzyl alcohol, chlorobutanol, o-cresol, phenyl ethyl alcohol; organic acids or salts thereof such as benzoic acid, sodium benzoate, potassium sorbate, parabens; or complex forming agents such as EDTA.

Excipients to adjust the tonicity of the composition may be added such as sodium chloride, glucose, dextrose, mannitol, sorbitol, lactose and the like. Additionally acid or basic buffers may be added to the intranasal composition to control the pH.

Pulmonary Dosage Forms

For pulmonary dosage forms acceptable other ingredients include, but are not limited to carriers and solvents such as but not limited to water, terpenes, alcohols such as ethanol, propylene glycol, glycerol, dimethylformamide, dimethylacetamide,. Other ingredients are phospholipids such as phosphatidylcholine, such as dipalmitoylphosphatidylcholihe, phosphatidylethanolamine, phosphatidylglycerol, phosphatidylserine. The phospholipids can be present in the formulation in the amount from about 0 to about 90 wt %. Preferably the phospholipids can be present in the composition in the amount from about 10 to about 60 wt. %. The formulation can include a surfactant, herein referring to any agent which preferentially absorbs to an interface between two immiscible phases, such as the interface between water and an organic polymer solution, a water/air interface or organic solvent/air interface. Surfactants may also promote absorption of a therapeutic or diagnostic agent and increase bioavailability of the agent. Suitable surfactants include but are not limited to hexadecanol; fatty alcohols such as polyethylene glycol (PEG); polyoxyethylene-9-lauryl ether; a surface active fatty acid, such as palinitic acid or oleic acid; glycocholate; surfactin; a poloxomer; a sorbitan fatty acid ester such as sorbitan trioleate (Span 85); and tyloxapol. The surfactant can be present in the composition in an amount ranging from about 0 to about 90 weight %. Preferably, it can be present in the composition in an amount ranging from about 10 to about 60 weight %.

In addition to lung surfactants, such as, for example, phospholipids discussed above, suitable surfactants include but are not limited to hexadecanol; fatty alcohols such as polyethylene glycol (PEG); polyoxyethylene-9-lauryl ether; a surface active fatty acid, such as palinitic acid or oleic acid; glycocholate; surfactin; a poloxomer; a sorbitan fatty acid ester such as sorbitan trioleate (Span 85); and tyloxapo

The composition can also optionally include an amino acid. Hydrophobic amino acids are preferred. Suitable amino acids include naturally occurring and non-naturally occurring hydrophobic amino acids. Examples of amino acids which can be employed include, but are not limited to: glycine, proline, alanine, 5 eysteine, methionine, valine, leucine′ tyro sine, isoleucine, phenylalanine, tryptophan.

The pulmonary composition can also include excipients such as, for example, dextran, polysaccliarides, trehalose, cyclodextrins, proteins, peptides, polypeptides, fatty acids, inorganic salts such as sodium chloride or calcium chloride, organic acid salts such as sodium citrate and phosphates such as sodium dihydrogen phosphate.

Although many such other ingredients are known to those skilled in the art, the inventors have found that only a sub-set of those provide for the most stable formulations. In particular, the inventors have found that preferred formulations contain less than about 20% w:w reducing carbohydrates. Reducing carbohydrates are sugars and their derivatives that contain a free aldehyde or ketone group capable of acting as a reducing agent through the donation of electrons. Examples of reducing carbohydrates include monosaccharides and disaccharides and more specifically include lactose, glucose, fructose, maltose and other similar sugars.

To ensure content uniformity of the blend, a volume mean diameter drug substance particle size of less than or equal to about 60 microns is preferably utilized. In one embodiment of the present invention, there is provided a composition and method for intranasal delivery of varenicline or its pharmaceutically acceptable salt thereof. Relative to an oral dosage form such as a tablet or capsule intranasal delivery of varenicline provides for rapid absorption, faster onset of therapeutic action and avoidance of liver first pass metabolism. For patients who have difficulty in swallowing tablets, capsules or other solids or those who have intestinal failure the intranasal delivery route of varenicline is preferred.

Generally, the composition for intranasal delivery includes varenicline or its pharmaceutically acceptable salts combined with various other ingredients known to those of skill in the art and as set forth above. Further, the composition can provide an initial rapid release of the active ingredient followed by a sustained release of the active ingredient. U.S. Pat. No. 5,629,011 provides examples of this type of formulation and is incorporated herein by reference in its entirety. There are numerous compositions that utilize intranasal delivery and related methods thereof. Moreover, there are numerous methods and related delivery vehicles that provide for intranasal delivery of various pharmaceutical compositions. For example, intranasal compositions that employ current marketed nicotine replacement therapies (See, N. J. Benowitz, Drugs, 45, 157-170, 1993, which is incorporated herein by reference in its entirety) are suitable for the active ingredient of the present invention. Further, polymer or cyclodextrin formulations can be used to enhance the bioavailability of the active ingredient of the present invention at the mucosal surface (See, U.S. Pat. Nos. 5,324,718 and 5,472,954; European Patent Number EP0579435 B1; and International Publication Numbers WO99/42111 and WO03/033025, all of which are incorporated herein by reference in their entirety).

Other nasal delivery compositions are chitosan-based and are suitable to increase the residence time of the active ingredient of the present invention on mucosal surfaces, which results in increasing its bioavailability. Examples of these nasal delivery compositions are disclosed in U.S. Pat. Nos. 6,465,626, 6,432,440, 6,391,318, and 5,840,341; European Patent Numbers EP0993483 and EP1051190; and International Publication Numbers WO 96/05810, WO 96/03142, and WO 93/15737, all of which are incorporated herein by reference in their entirety.

Intranasal compositions according to the invention can be administered by any appropriate method according to their form. A composition including microspheres or a powder can be administered using a nasal insufflator device. Examples of these devices are already employed for commercial powder systems intended for nasal application (e.g., Fisons Lomudal System). The insuflator produces a finely divided cloud of the dry powder or microspheres. The insufflator is preferably provided with a mechanism to ensure administration of a substantially fixed amount of the composition. The powder or microspheres can be used directly with an insufflator, which is provided with a bottle or container for the powder or microspheres. Alternatively, the powder or microspheres can be filled into a capsule such as a gelatin capsule, or other single dose device adapted for nasal administration. The insufflator preferably has a mechanism to break open the capsule or other device.

Relative to an oral dosage form such as a tablet or capsule intranasal delivery of varenicline provides for rapid absorption, faster onset of therapeutic action and avoidance of liver first pass metabolism. For patients who have difficulty in swallowing tablets, capsules or other solids or those who have intestinal failure the intranasal delivery route of varenicline is preferred. Further, the composition can provide an initial rapid release of the active ingredient followed by a sustained release of the active ingredient.

A composition including a solution or dispersion in an aqueous medium can be administered as a spray for intranasal delivery of the active ingredient by using an appropriate device such as a metered dose aerosol valve or a metered dose pump. A gas or liquid propellant can be used. Details of other devices are disclosed in the following patents, patent applications and publications: WO03/026559, WO02/11800, WO00/51672, WO02/068029, WO02/068030, WO02/068031, WO02/068032, WO03/000310, WO03/020350, WO03/082393, WO03/084591, WO03/090812, WO00/41755, and the pharmaceutical literature (See, Bell, A. Intranasal Delivery Devices, in Drug Delivery Devices Fundamentals and Applications, Tyle P. (ed), Dekker, New York, 1988), Remington's Pharmaceutical Sciences, Mack Publishing Co., 1975, all of which are incorporated herein by reference in their entirety.

Transport of the active ingredient of the present invention across normal mucosal surfaces such as the nasal, pulmonary, buccal or sublingual mucosa, can be enhanced by optionally combining it with an absorption promoting agent such as those disclosed in U.S. Pat. Nos. 5,629,011, 5,023,252, 6,200,591, 6,369,058, 6,380,175, and International Publication Number WO 01/60325, all of which are incorporated herein by reference in their entirety. Examples of these absorption promoting agents include, but are not limited to, cationic polymers, surface active agents, chelating agents, mucolytic agents, cyclodextrin, polymeric hydrogels, combinations thereof, and any other similar absorption promoting agents known to those of skill in the art.

Polymeric hydrogel-forming compositions, which exhibit muco-adhesion and controlled drug release properties, can also be optionally included in the intranasal compositions of the present invention. Examples of such formulations are disclosed in U.S. Pat. Nos. 6,068,852 and 5,814,329; and International Publication Number WO99/58110, all of which are incorporated herein by reference in their entirety. Additionally, the present invention can be formulated with powder microsphere and mucoadhesive compositions as disclosed in European Patent Numbers EP1025859 and EP1108423, all of which are incorporated herein by reference in their entirety. Finally, thiolated polymeric excipients that form covalent bonds with the cysteine rich subdomains of the mucus membrane can also provide mucoadhesion, which prolongs the contact time between the active ingredient and the membrane. Such excipients are disclosed in International Publication Number WO03/020771, which is incorporated herein by reference in its entirety.

In another embodiment of the present invention, there is provided a composition including a solid dosage form for transmucosal, and preferably buccal or sublingual delivery. The solid dosage form can be immediate release or controlled release, wherein the dosage form disintegrates and/or melts in the oral cavity at body temperature with or without the aid of fluids, salivary fluids, mechanical erosion, or combinations thereof. Alternatively, the dosage form can be sprayed into the oral cavity in the form of a solution spray or a dry powder. Relative to an oral dosage form such as a tablet or capsule buccal delivery of varenicline provides for rapid absorption, faster onset of therapeutic action and avoidance of liver first pass metabolism. For patients who have difficulty in swallowing tablets, capsules or other solids or those who have intestinal failure the buccal or sublingual delivery route of varenicline is preferred. Further, the composition can provide an initial rapid release of the active ingredient followed by a sustained release of the active ingredient. Generally, the composition may be adhesive towards the body tissue lining the oral cavity of the individual. The dosage form can be, but is not limited to, tablets, a bioadhesive patch or film, lozenges, hard candies, wafers, lollipops, sprays, gums, pills, pellets, spheres, combinations thereof, and other forms known to those of skill in the art.

There are numerous compositions and delivery vehicles suitable for buccal or sublingual delivery of the active ingredient of the present invention. Examples of such compositions or delivery vehicles are disclosed in U.S. Pat. Nos. 6,676,959, 6,676,931, 6,593,317, 6,552,024, 6,306,914, 6,284,264, 6,248,358, 6,210,699, 6,177,096, 6,197,331, 6,153,222, 6,126,959, 6,286,698, 6,264,981, 6,187,323, 6,173,851, 6,110,486, 5,955,098, 5,869,082, 5,985,311, 5,948,430, 5,753,256, 5,487,902, 5,470,566, 5,362,489, 5,288,498, 5,288,497, 5,269,321, 6,488,953, 6,126,959, 6,641,838, 6,576,250, 6,509,036, 6,391,335, 6,365,182, 6,280,770, 6,221,392, 6,200,604, 6,531,112, and 6,485,706, all of which are incorporated herein by reference in their entirety.

In a further embodiment of the present invention, there is provided a composition and related method for pulmonary delivery. Relative to an oral dosage form such as a tablet or capsule buccal delivery of varenicline provides for rapid absorption, faster onset of therapeutic action and avoidance of liver first pass metabolism. For patients who have difficulty in swallowing tablets, capsules or other solids or those who have intestinal failure the pulmonary delivery route of varenicline is preferred. Further, the composition can provide an initial rapid release of the active ingredient followed by a sustained release of the active ingredient.

Compositions for pulmonary delivery according to the present invention can be administered by any appropriate method according to their form. Preferably, particles administered to the respiratory tract travel through the upper airways (oropharyiix and larynx), the lower airways which include the trachea followed by bifurcations into the bronchi and bronchioli and through the tenninal bronchioli which in turn divide into respiratory bronchioli leading then to the ultimate respiratory zone, the alveoli or the deep lung. In a preferred embodiment of the invention, most of the mass of particles deposits in the deep lung or alveoli. This includes solutions or dispersions in an aqueous medium can be administered as a spray with an atomizer to create droplets with narrow size distributions. A composition including microspheres or a powder can be administered using a nasal insufflator device herein mentioned above. The insufflator produces a finely divided cloud of the dry powder or microspheres. The insufflator is preferably provided with a mechanism to ensure administration of a substantially fixed amount of the composition. The powder or microspheres can be used directly with an insufflator, which is provided with a bottle or container for the powder or microspheres. Alternatively, the powder or microspheres can be filled into a capsule such as a gelatin capsule, or other single dose device adapted for nasal administration. The insufflator preferably has a mechanism to break open the capsule or other device.

Other suitable devices are disclosed in U.S. Pat. No. 5,797,392, European Patent Number EP 0805696, and International Publication Number WO 02/078774, all of which are incorporated herein by reference.

Other features and embodiments of the invention will become apparent from the following examples, which are given for illustration of the invention rather than for limiting its intended scope.

### EXAMPLES

### Example 1

A liquid formulation is prepared by dissolving 150 mg of varenicline tartrate in 10 ml of a 0.5% solution of medium viscosity grade of Chitosan (80% degree of deacetylation obtained from Protan Limited). The substituted cyclodextrin material beta-cyclodextrin (Sigma Chemical, Co.) is added to provide a concentration of 5%. The liquid formulation is administered to the nose using a conventional pump spray device.

### Example 2

A bioadhesive powder formulation of varenicline tartrate is prepared using microspheres of cross-linked starch. The microspheres are prepared by the method described in GB 1518121 and EP 223302 described above and incorporated herein by reference in their entirety. A preferred size of microspheres is 1-100 micrometers.

75 mg of varenicline tartrate is dissolved in 30 ml water and mixed with 1 g of starch microspheres. The product is freeze-dried to produce a free flowing powder. The final concentration of varenicline tartrate in the product is 0.05 mg/mg of starch microspheres. The powder is administered to the nasal cavity using an insufflator device. The quantity administered is 1.0 mg microspheres per kg body weight containing 0.05 mg varenicline tartrate.

### Example 3

A nasal spray solution is exemplified herein. Varenicline tartrate (1.5 g) is dissolved in 100 ml 0.05M Phosphate buffer (pH 4.4) and sufficient sodium chloride is added to the solution to make it isotonic. The solution is placed in a nasal administrator designed to deliver 100 μl of spray for each application. One spray in each nostril delivers a total of 3 mg of varenicline tartrate (approximately equivalent to1.7 mg varenicline free base).

### Example 4

An aqueous nasal gel is exemplified herein. The aqueous nasal gel includes the following components:

| | | |
|---|---|---|
| Varenicline free base | 1 | g |
| Methocel | 3 | g |
| 0.05M Acetate buffer, pH 4.4 | 100 | g |

Approximately 70 g of buffer is heated to 80° C., and the methocel is dispersed in it with stirring. The varenicline free base is dissolved in 30 g of buffer at 80° C., and the solution is mixed with the methocel dispersion. The resultant mixture is allowed to stand at room temperature for three hours. An appropriate amount of gel is placed in an ointment tube equipped with a fine orifice and is applied in the nasal nares with a finger or cotton tipped applicator.

### Example 5

Chitosan (6 mg, 0.6% w/w) is suspended in demineralized water. Lactic acid (4 mg, 0.4% w/w) is added while stirring until a clear solution was obtained. Benzalkonium chloride (0.1 mg, 0.01% w/w) is also added. The pH of the solution is brought to 5.5 by adding a 0.01 N solution of sodium hydroxide. The active ingredient, i.e. varenicline free base (12.4 mg, 1.24% w/w) is dissolved in the stirred solution. Demineralized water is added to obtain a final volume of 1 ml. Sodium chloride then is added (5 mg, 0.5% w/w). The resulting solution is stored in small vials of about 125 micro liters. These vials are adapted to fit into one unidose spraying device, which delivers about 100 micro liters of solution. Each dose of 100 microliters contains approximately 1.2 mg of varenicline free base.

### Example 6

Varenicline tartrate is ground in a mortar to an average particle size of 5 μm. To 6 mg of this varenicline tartrate, 180 mg of microcrystalline cellulose (FMC Biopolymer, Philadelphia, Pa., Avicel PH101 with a 90% w/w average particles size of 63 to 150 μm) and 20 mg of hydroxypropyl cellulose (Aqualon, Wilmington, Del.: HPC-H with a 90% w/w average particles size of 10 to 100 μm) are added. Then, as a lubricant, 0.16 mg of magnesium stearate is added to form a mixture. The resulting mixture is blended to prepare a powder composition.

### Example 7

A solid dosage form for buccal absorption is produced by melting 67.6 gm Akoprime E (a hydrogenated vegetable oil type II NF 18, Karlshamns, AB, Karlshamn, Sweden), at 40° C. until a clear solution is obtained. At 40° C., xylitol (10 gm), cacao (16.9 gm), and a flavoring agent are added in portions and under homogenization until a visually dispersed system is obtained after which varenicline free base (0.25 gm) is added. At 40° C., the mixture was further homogenized after which 5 gm egg lecithin (purified egg phospholipids) is added under continued homogenization. The obtained mixture is dispensed and molded in blisters and cooled to room temperature resulting in pieces ranging from 0.4 to about 0.8 g. There is 3.13 mg of varenicline per 0.8 gm dosage form.

### Example 8

### Preparation of a Pulmonary Dry Powder Composition of Varenicline

Varenicline free base( 0.01 g ) and 2.0 g dipalmitoylphosphatidylcholine (DPPC) (Avanti Polar Lipids,) are dissolved in 2.80 L of ethanol. To this is added 0.91 g Sodium Citrate (Dihydrate) and 0.53 g Calcium Chloride (Dihydrate) are added to 1.2 L of water and dissolved. The two solutions are combined by adding the water solution to the ethanol solution and then the solutions are allowed to stir until the solution is clear. The final solution was then spray dried in a Niro dryer (Niro, Inc., Columbus, Md.) using a rotary atomizer and nitrogen drying gas following the direction of the manufacturer, using the following spray conditions: Ti,′j.,=12TC, T.Utlet=54′C, feed rate=65 inl/min, heat nitrogen=38 mm. H20, atomizer speed=20,000 rpm (V24 atomizer used). The weight percent of the dry powder formulation was approximately: 1% varenicline, 58% DPPC, 26% Sodium Citrate, 15% Calcium Chloride.

### Example 8

### Preparation of a Pulmonary Aerosol Solution Composition of Varenicline

Varenicline tartrate (0.01 g) is dissolved in 50 mL of ethanol. To this solution is dissolved 0.01 g of ascorbic acid. The resulting solution can be used in loaded into an aerosol metered dose inhalation device charged with a suitable propellant such as dichlorodifluormethane or dichlortetrafluoroethane.

Throughout this application, various publications, including U.S. patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Many modifications and variations of the present invention are possible in light of the above teachings. It is, therefore, to be understood that within the scope of the appended claims, the invention can be practiced otherwise than as specifically described.

What is claimed is:

1. A composition for nasal administration comprising varenicline or its pharmaceutically acceptable salt.

2. The composition according to claim 1, wherein said varenicline or its pharmaceutically acceptable salt is in a dosage range of 0.1 mgA to 6 mgA per day.

3. The composition according to claim 1, wherein said pharmaceutically acceptable salt is varenicline tartrate.

4. The composition according to claim 1, further comprising an absorption promoting agent is selected from the group consisting of a cationic polymer, a surface active agent, a chelating agent, a mucolytic agent, a cyclodextrin, polymeric hydrogel, and combinations thereof.

5. The composition according to claim 1 further comprising excipients, diluents, binders, lubricants, glidants, disintegrants, carriers, surfactants, flavors and mixtures thereof.

6. The composition according to claim 1, wherein the composition is in a form selected from the group consisting of a liquid, liquid spray, microspheres, and powder.

7. A composition for buccal or sublingual administration comprising varenicline or its pharmaceutically acceptable salt in a solid dosage form, wherein said solid dosage form disintegrates in an oral cavity at body temperature, and may adhere to the body tissue of the oral cavity.

8. The composition according to claim 7, wherein said varenicline or its pharmaceutically acceptable salt is in a dosage range of 0.1 mgA to 6 mgA per day.

9. The composition according to claim 7, wherein said varenicline or its pharmaceutically acceptable salt includes varenicline tartrate.

10. The composition according to claim 7, further comprising at least one ingredient selected from the group consisting of excipients, diluents, binders, lubricants, glidants, disintegrants, carriers, surfactants, flavors and mixtures thereof.

11. The composition according to claim 7, wherein the composition is in a form selected from the group consisting of a tablet, pill, bioadhesive patch, film, lozenges, hard candy, wafers, sphere, lollipop, disc-shaped structure, and spray.

12. A composition for pulmonary administration comprising varenicline or its pharmaceutically acceptable salt.

13. The composition according to claim 12, wherein said varenicline or its pharmaceutically acceptable salt is in a dosage range of 0.1 mgA to 6 mgA per day.

14. The composition according to claim 13, wherein said pharmaceutically acceptable salt is varenicline tartrate.

15. The composition according to claim 12, further comprising at least one ingredient selected from the group consisting of excipients, diluents, binders, lubricants, glidants, disintegrants, carriers, surfactants, flavors and mixtures thereof.

16. A method for reducing nicotine addiction, aiding in the cessation of, or lessening of, tobacco use in a subject comprising administering to the subject an effective amount of the composition in accordance with claims 1, 7, or 12.

17. An intranasal, buccal, sublingual or pulmonary dosage form suitable for administration to a subject comprising a core containing 5,8,14-triazatetracyclo[10.3.1.0^{2,11}.0^{4,9}]-hexadeca-2(11),3,5,7,9-pentaene, or a pharmaceutically acceptable salt thereof, and pharmaceutically acceptable excipient(s), wherein the total level of reducing carbohydrates is less than 20 wt %.
